(19) United States
(12) Patent Application Publication
(10) Pub. No.: US 2026/0014393 A1
(43) Pub. Date: Jan. 15, 2026

(54) METHODS, SYSTEMS, AND COMPUTER READABLE MEDIA FOR THREE-DIMENSIONAL PRINTING OF MODULAR HETEROGENEOUS DENSITY DEVICES FOR RANGE MODULATION IN PARTICLE THERAPY

(71) Applicant: The Trustees of the University of Pennsylvania, Philadelphia, PA (US)

(72) Inventors: Boon-Keng Kevin Teo, Wayne, PA (US); Peter Benjamin Theodor Noel, Narbeth, PA (US); Wei Zou, Skillman, NJ (US); Wenbo Gu, Philadelphia, PA (US); Alexander Lin, Swarthmore, PA (US); Lei Dong, Philadelphia, PA (US); Kai Mei, Philadelphia, PA (US)

(21) Appl. No.: 19/269,743

(22) Filed: Jul. 15, 2025

Related U.S. Application Data
(60) Provisional application No. 63/671,715, filed on Jul. 15, 2024.

(51) Int. Cl. A61N 5/10

(52) U.S. Cl. CPC A61N 5/1065

(57) ABSTRACT

A method for generating a range modulating device for radiotherapy includes obtaining a medical image of a patient. The method further includes determining, using the medical image, a design of a range modulating device having non-uniform density for radiotherapy for the patient. The method further includes 3D printing, using the design, the range modulating device by continuously varying the ratio of filament to air in each voxel of a plurality of voxels. The method can also include performing radiotherapy on the patient by inserting the range modulating device in a beam path between a beam from a radiation source and the patient.

### PRIORITY CLAIM

This application claims the priority benefit of U.S. Provisional Patent Application Ser. No. 63/671,715, filed Jul. 15, 2024, the disclosure of which is incorporated herein by reference in its entirety.

### TECHNICAL FIELD

The subject matter described herein relates generally to methods, systems, and computer readable media for three-dimensional printing of modular heterogeneous density devices for range modulation in particle therapy.

### BACKGROUND

Particle radiation therapy utilizing high energy charged particles, such as protons or carbon ions, offer an advantage over conventional high energy X-ray radiation therapy for cancer treatments due to its characteristic depth dose deposition profile, known as the Bragg peak. The finite range of particle beams (such as protons, carbons and electrons) is dependent on the beam energy, which leads to a sharp distal dose fall-off (Bragg peak) and which permits lower radiation to be received by normal tissue distal to the cancer target along the beam path. Pencil beam scanning (PBS) particle therapy is an implementation of particle therapy utilizing pristine mono-energetic pencil beams with a narrow lateral and depth dose profile. PBS particle therapy utilizes electro-magnets to scan the beam laterally while depth coverage is achieved by sweeping the pencil beam energy.

Conventional PBS delivery can take from several seconds to tens of seconds to deliver a radiation field that consists of several energy layers. For a cyclotron based system, energy selection is accomplished by varying the thickness of energy degrading material to reduce the particle beam energy after it emerges from the cyclotron. There are benefits to faster radiation delivery namely (1) motion management, whereby reducing the treatment time below a single breath hold can reduce the volume of irradiated normal tissue as well as reduce dose heterogeneity effects due to the interplay of target motion and the pencil beam delivery (2) exploit the enhanced normal tissue sparing afforded by ultra-high dose rate delivery due to the FLASH effect. Faster delivery can be accomplished by increasing the pencil beam intensity and/or reducing the number of energy layers required for volumetric irradiation down to one or several energies.

### SUMMARY

According to one aspect of the subject matter described herein, a method includes obtaining a medical image of a patient. The method further includes determining, using the medical image, a design of a range modulating device having non-uniform density for radiotherapy for the patient. The method further includes 3D printing, using the design, the range modulating device by continuously varying the ratio of filament to air in each voxel of a plurality of voxels.

According to another aspect of the subject matter described herein, the method includes performing radiotherapy on the patient by inserting the range modulating device in a beam path between a beam from a radiation source and the patient.

According to another aspect of the subject matter described herein, performing radiotherapy comprises scanning the beam laterally using at least one magnetic field.

According to another aspect of the subject matter described herein, determining the design of the range modulating device comprises designing the range modulating device to have a spatially varying thickness and density of energy degrading material across a transverse plane of the beam such that performing radiotherapy by inserting the range modulating device in the beam path creates a spread out Bragg peak (SOBP).

According to another aspect of the subject matter described herein, determining the design of the range modulating device comprises identifying a tumor in the medical image and designing the range modulating device to obtain, while performing radiotherapy, a radiation dose distribution suitable for a shape of the tumor.

According to another aspect of the subject matter described herein, performing radiotherapy comprises administering FLASH irradiation for a duration of one second or shorter.

According to another aspect of the subject matter described herein, the method includes assembling a composite range modulator from the range modulating device and at least one other prefabricated modular range modulating device.

According to another aspect of the subject matter described herein, assembling a composite range modulator comprises stacking parallel to or perpendicular to the particle beam or both.

According to another aspect of the subject matter described herein, determining the design of the range modulating device comprises converting the medical image into fused deposition modeling (FDM) printer instructions.

According to another aspect of the subject matter described herein, determining the design of the range modulating device comprises modeling density as a ratio of filament to voxel volume to emulate attenuation profiles for each voxel. More than one filament type, including metal filaments, may be used.

According to another aspect of the subject matter described herein, a system includes a computer system programmed for obtaining a medical image of a patient and determining, using the medical image, a design of a range modulating device having non-uniform density for radiotherapy for the patient. The system further includes a 3D printer configured for printing, using the design, the range modulating device by continuously varying the ratio of filament to air in each voxel of a plurality of voxels.

According to another aspect of the subject matter described herein, the system further includes an irradiation system configured for performing radiotherapy on the patient by generating a beam passing through the range modulating device in a beam path to the patient.

According to another aspect of the subject matter described herein, the system includes a composite range modulator assembled from the range modulating device and at least one other prefabricated modular range modulating device.

According to another aspect of the subject matter described herein, the composite range modulator is stacked parallel to or perpendicular to the particle beam direction or both.

According to another aspect of the subject matter described herein, determining the design of the range modulating device comprises modeling density as a ratio of filament to voxel volume to emulate attenuation profiles for each voxel.

According to another aspect of the subject matter described herein, a method includes assembling a composite range modulator by stacking a first range modulating device and a second range modulating device. The method further includes performing radiotherapy on a patient by inserting the composite range modulator into a beam path between a radiation source and the patient.

According to another aspect of the subject matter described herein, assembling the composite range modulator comprises stacking parallel to or perpendicular to the particle beam or both.

According to another aspect of the subject matter described herein, a range modulating device is produced by the method described above, and the range modulating device includes a plurality of spikes of energy shifting material having non-uniform density.

The subject matter described herein may be implemented in hardware, software, firmware, or any combination thereof. As such, the terms “function” or “node” as used herein refer to hardware, which may also include software and/or firmware components, for implementing the feature(s) being described. In some exemplary implementations, the subject matter described herein may be implemented using a computer readable medium having stored thereon computer executable instructions that when executed by the processor of a computer control the computer to perform steps. Exemplary computer readable media suitable for implementing the subject matter described herein include non-transitory computer readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

Exemplary implementations of the subject matter described herein will now be explained with reference to the accompanying drawings, of which:

FIG. 1A shows an example of a 3D printed range modulator that produces a 2 cm SOBP from a monoenergetic particle beam;

FIG. 1B shows an axial view of a CT scan of the modulator;

FIG. 1C shows a sagittal view of the CT scan;

FIG. 1D is a density profile across the heterogenous density range modulating device;

FIGS. 2A and 2B illustrate creating a composite range modulator stacked in an arrangement parallel (FIG. 2A) to or perpendicular (FIG. 2B) to the particle beam;

FIG. 2C shows the spread out Bragg peak (SOBP) of a 2 cm modulator;

FIG. 2D shows the SOBP of a 3 cm modulator;

FIG. 2E shows the SOBP of a stacked composite of 2 cm and 3 cm modulators with 5 cm SOBP calculated using Monte Carlo simulation;

FIG. 3 illustrates an example of a range modulator design having uniform density spikes that can be manufactured using a traditional 3D printer; and

FIG. 4 is a block diagram of an example system for three-dimensional printing of modular heterogeneous density devices for range modulation in particle therapy.

### DETAILED DESCRIPTION

Beam shaping and range modulating devices are passive components inserted in the beam path to modify the depth dose characteristics in order to create a spread-out Bragg peak (SOBP). These work by spatially varying the thickness of energy degrading material across the transverse plane of the particle beam, thereby creating a distribution of particle energies that give rise to a SOBP. These devices can be constructed using machining techniques or 3D printing.

The subject matter described herein includes a class of range modulating devices for particle therapy. The range modulating devices are 3D printed using spatially modulated density printing technology. During the 3D printing of the range modulating devices, by continuously varying the ratio of filament to air in each voxel, 3D structures with spatially modulated density are used to create a SOBP. The range modulation is thus accomplished by both material density modulation as well as thickness of material, while existing designs modulate only the thickness of materials with fixed density binary 3D printing.

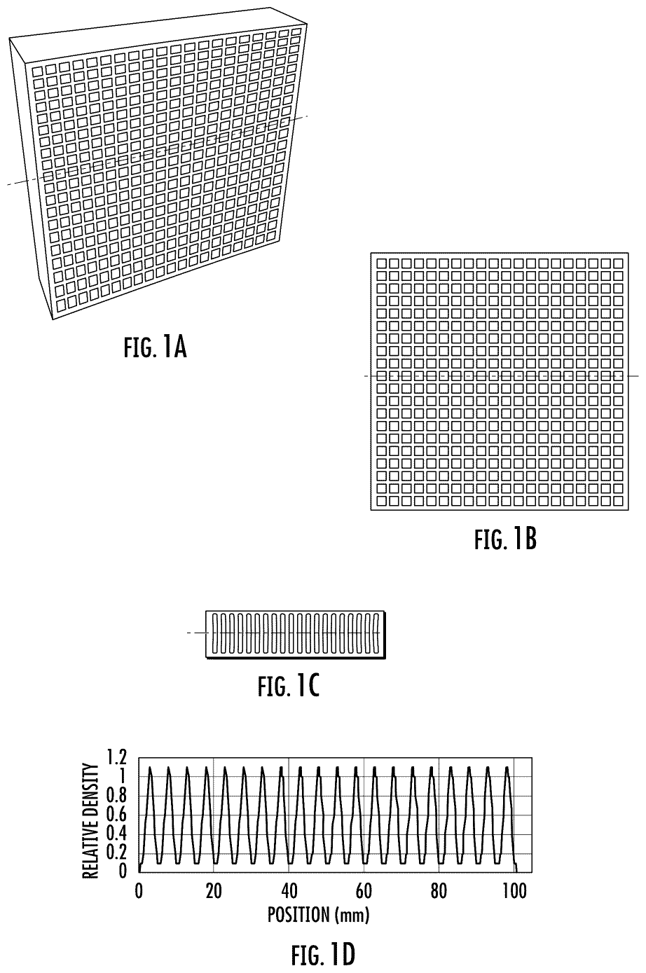

FIG. 1A shows an example of a 3D printed range modulator that produces a 2 cm SOBP from a monoenergetic particle beam. FIG. 1B shows an axial view of a CT scan of the modulator. FIG. 1C shows a sagittal view of the CT scan. FIG. 1D is a density profile across the heterogenous density range modulating device.

As shown in FIG. 1D, the transverse density profile varies from 0.1 g/cm³ for regions with low fill ratio to 1.1 g/cm³ which corresponds to the density of the material used for 3D printing. Higher density regions can be achieved by utilizing metallic filaments. Patient specific range modulators with spatially varying range modulation can by generated by using an iterative optimization technique similar to 3D medical image reconstruction which comprises iteration between the forward calculation of the particle beam propagating through the range modulator followed by update of the density map of the range modulator.

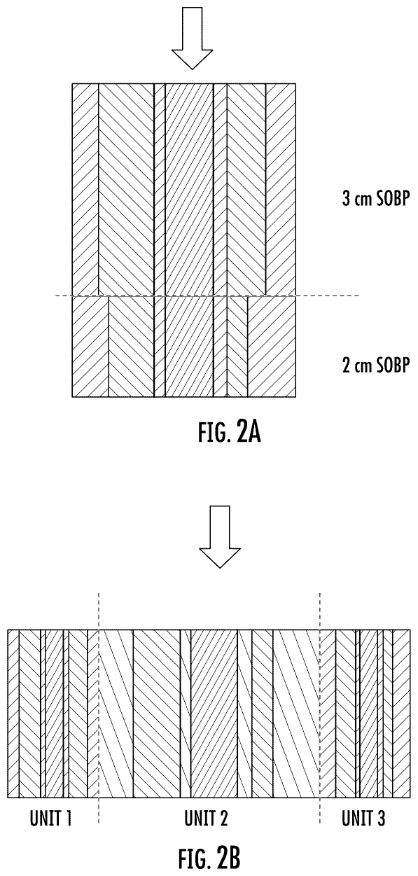

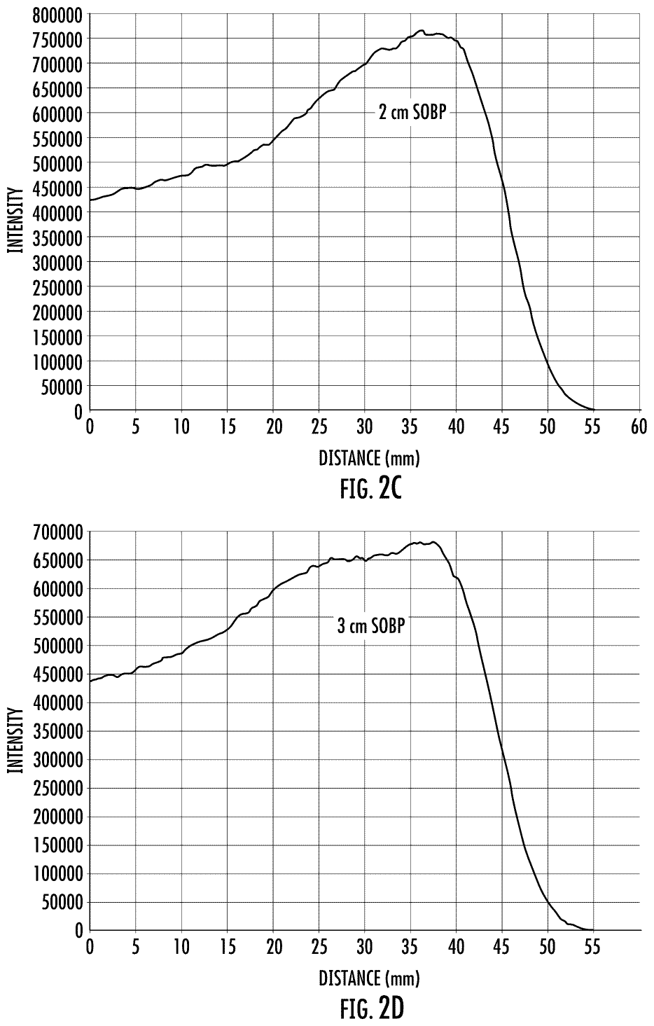

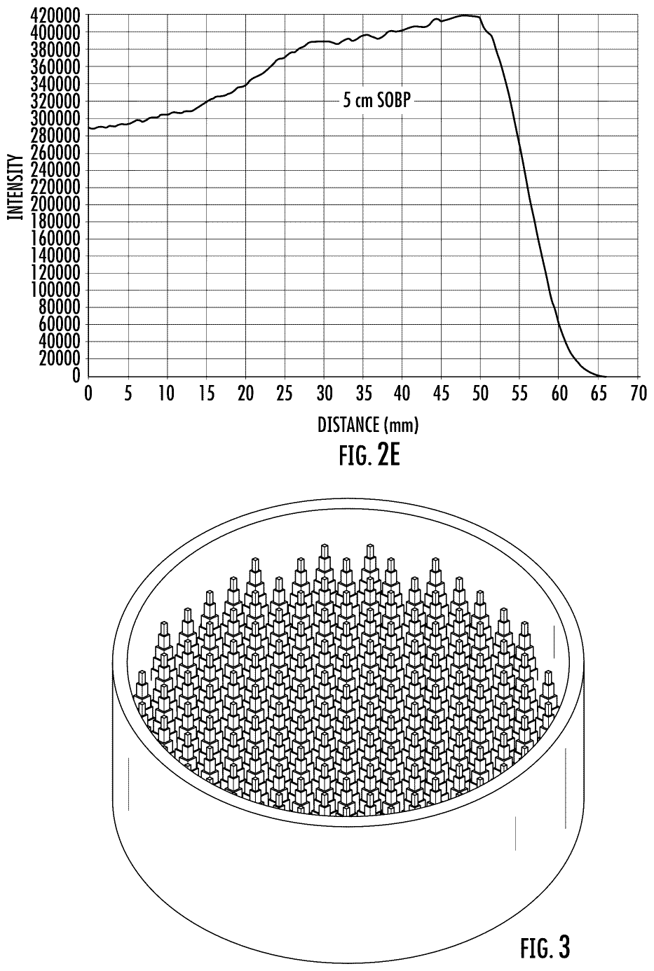

Composite range modulators can be constructed from smaller modulators via stacking of smaller modulator units. For example, FIGS. 2A-2B illustrate creating a composite range modulator via stacking parallel to (FIG. 2A) and perpendicular (FIG. 2B) to the particle beam direction. The arrows indicate the particle beam irradiation direction. FIG. 2C shows the SOBP of a 2 cm modulator, FIG. 2D shows the SOBP of a 3 cm modulator, and FIG. 2E shows the SOBP of a stacked composite of 2 cm and 3 cm modulators with 5 cm SOBP calculated using Monte Carlo simulation.

FIG. 2A demonstrates stacking of a 2 cm SOBP and 3 cm SOBP modulator pair parallel to particle beam direction to produce an equivalent 5 cm SOBP. Spatially modulated range modulators can be constructed by laterally assembling several modulator units (FIG. 2B) to create a larger SOBP at the center but smaller SOBP at the peripheral. Such modular devices may be used in place of printing a patient specific modulator, permitting quick assembly from a library of small modulator units.

The systems and methods described herein can be used for fabricating range modulators for particle therapy in a way that enables faster treatments by delivering radiation from particle therapy systems to be spread out in the beam direction while being scanned laterally using magnetic fields. FIG. 3 (from Titt et al. Med Phys.2022; 49:497-50; https://doi.org/10.1002/mp.15370) shows an example of a range modulator design having uniform density spikes that can be manufactured using a traditional 3D printer. Due to limitations of 3D printing, the height of these spikes is limited by the stability of the spikes as they need to be self-supporting. The range modulator devices described herein have non-uniform density and can be made to be modular, permitting composite range modulators to be assembled and stacked both vertically as well as laterally.

These devices can be designed and manufactured for each patient's particle therapy treatment specifications. It will be mounted on the particle therapy system delivery beam nozzle and will be used to reduce treatment time based on either (1) the need to reduce the irradiated volume for the treatment of moving targets and/or (2) exploit the benefit of the FLASH effect to spare normal tissue arising from ultra-fast irradiation. In some situations, a composite range modulator can be assembled from prefabricated modular units by stacking in both vertical and horizontal directions.

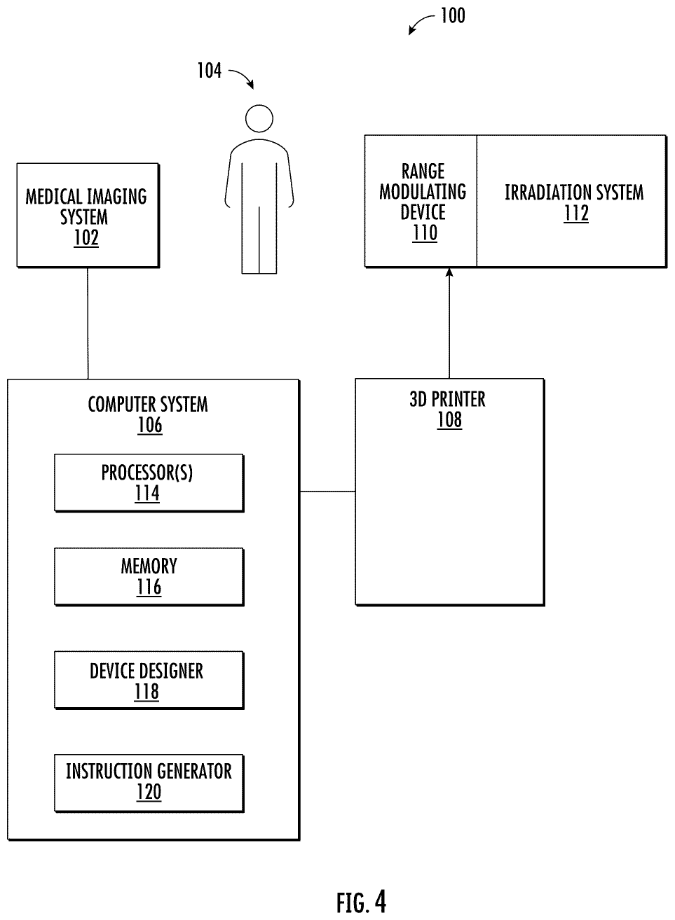

FIG. 4 is a block diagram of an example system 100 for three-dimensional printing of modular heterogeneous density devices for range modulation in particle therapy.

Referring to FIG. 4, a system 100 includes a medical imaging system 102 for taking a medical image of a patient 104. The medical imaging system 102 can be any suitable medical imaging system, such as a magnetic resonance imaging (MRI) system or a computed tomography (CT) imaging system.

System 100 includes a computer system 106 configured for obtaining the medical image and determining, using the medical image, a design of a range modulating device 110 having non-uniform density for radiotherapy for the patient 104. Computer system 106 can obtain the image directly from the medical imaging system 102 or from another appropriate source, e.g., over a data communications network.

System 100 includes a 3D printer 108 configured for printing, using the design, range modulating device 110 by continuously varying the ratio of filament to air in each voxel of a number of voxels.

System 100 includes an irradiation system 112 configured for performing radiotherapy on the patient 104 by generating a beam passing through the range modulating device 110 in a beam path to patient 104. Performing radiotherapy can include scanning the beam laterally using at least one magnetic field. Performing radiotherapy can include administering FLASH irradiation for a duration of one second or shorter.

Computer system 106 includes at least one processor 114 and memory 116 storing instructions executed by processor 114. Computer system 106 includes a device designer 118 for designing range modulating device 110 and an instruction generator 120 for generating 3D printing instructions, e.g., fused deposition modeling (FDM) printer instructions, such as G code. In some examples, device designer 118 and instruction generator 120 are implemented on separate computer systems.

In some examples, images representing various densities of the original patient data are directly converted into FDM printer instructions (G-code). In order to emulate attenuation profiles for each voxel, density is modeled as a ratio of filament to voxel volume (generating partial volume effects) with the filament ratio being continuously modified by varying the printing speed. Filament lines in adjacent layers are shifted in position and arranged in alternating directions (90-degree rotations) to reduce patterns caused by stacking the infill lines directly above one another. The varying line widths create a two-dimensional matrix that correlates with the original 3D input image volume. Because the filament lines in this matrix are smaller than the resolution limit of clinical CT scans, they generate partial volume effects which result in representative HU values.

Device designer 118 can be configured for identifying a tumor in the medical image and designing the range modulating device to obtain, while performing radiotherapy, a radiation dose distribution suitable for a shape of the tumor. For example, device designer 118 can use Monte Carlo simulations in conjunction with empirical iterative adjustments in shapes, sizes, and positions to characterize the incident beam and design the range modulating device 110.

Device designer 118 can be configured for designing the range modulating device to have a spatially varying thickness and density of energy degrading material across a transverse plane of the beam such that performing radiotherapy by inserting the range modulating device in the beam path creates a spread out Bragg peak (SOBP). Determining the design of the range modulating device comprises modeling density as a ratio of filament to voxel volume to emulate attenuation profiles for each voxel.

Although specific examples and features have been described above, these examples and features are not intended to limit the scope of the present disclosure, even where only a single example is described with respect to a particular feature. Examples of features provided in the disclosure are intended to be illustrative rather than restrictive unless stated otherwise. The above description is intended to cover such alternatives, modifications, and equivalents as would be apparent to a person skilled in the art having the benefit of this disclosure.

The scope of the present disclosure includes any feature or combination of features disclosed in this specification (either explicitly or implicitly), or any generalization of features disclosed, whether or not such features or generalizations mitigate any or all of the problems described in this specification. Accordingly, new claims may be formulated during prosecution of this application (or an application claiming priority to this application) to any such combination of features. In particular, with reference to the appended claims, features from dependent claims may be combined with those of the independent claims and features from respective independent claims may be combined in any appropriate manner and not merely in the specific combinations enumerated in the appended claims.

What is claimed is:

1. A method comprising:
obtaining a medical image of a patient;
determining, using the medical image, a design of a range modulating device having non-uniform density for radiotherapy for the patient; and
3D printing, using the design, the range modulating device by continuously varying a ratio of filament to air in each voxel of a plurality of voxels.

2. The method of claim 1, comprising performing radiotherapy on the patient by inserting the range modulating device in a beam path between a beam from a radiation source and the patient.

3. The method of claim 2, wherein performing radiotherapy comprises scanning the beam laterally using at least one magnetic field.

4. The method of claim 2, wherein determining the design of the range modulating device comprises designing the range modulating device to have a spatially varying thickness and density of energy degrading material across a transverse plane of the beam such that performing radiotherapy by inserting the range modulating device in the beam path creates a spread out Bragg peak (SOBP).

5. The method of claim 2, wherein determining the design of the range modulating device comprises identifying a tumor in the medical image and designing the range modulating device to obtain, while performing radiotherapy, a radiation dose distribution suitable for a shape of the tumor.

6. The method of claim 2, wherein performing radiotherapy comprises administering FLASH irradiation for a duration of one second or shorter.

7. The method of claim 1, comprising assembling a composite range modulator from the range modulating device and at least one other prefabricated modular range modulating device.

8. The method of claim 7, wherein assembling a composite range modulator comprises stacking parallel to a particle beam direction, perpendicular to the particle beam direction, or parallel and perpendicular to the particle beam direction.

9. The method of claim 1, wherein determining the design of the range modulating device comprises converting the medical image into fused deposition modeling (FDM) printer instructions.

10. The method of claim 1, wherein determining the design of the range modulating device comprises modeling density as a ratio of filament to voxel volume to emulate attenuation profiles for each voxel.

11. A system comprising:
a computer system programmed for obtaining a medical image of a patient and determining, using the medical image, a design of a range modulating device having non-uniform density for radiotherapy for the patient; and
a 3D printer configured for printing, using the design, the range modulating device by continuously varying a ratio of filament to air in each voxel of a plurality of voxels.

12. The system of claim 11, comprising an irradiation system configured for performing radiotherapy on the patient by generating a beam passing through the range modulating device in a beam path to the patient.

13. The system of claim 12, wherein performing radiotherapy comprises scanning the beam laterally using at least one magnetic field.

14. The system of claim 12, wherein determining the design of the range modulating device comprises designing the range modulating device to have a spatially varying thickness and density of energy degrading material across a transverse plane of the beam such that performing radiotherapy by inserting the range modulating device in the beam path creates a spread out Bragg peak (SOBP).

15. The system of claim 12, wherein determining the design of the range modulating device comprises identifying a tumor in the medical image and designing the range modulating device to obtain, while performing radiotherapy, a radiation dose distribution suitable for a shape of the tumor.

16. The system of claim 12, wherein performing radiotherapy comprises administering FLASH irradiation for a duration of one second or shorter.

17. The system of claim 11, comprising a composite range modulator assembled from the range modulating device and at least one other prefabricated modular range modulating device.

18. The system of claim 17, wherein the composite range modulator is stacked parallel to a particle beam direction, perpendicular to the particle beam direction, or parallel and perpendicular to the particle beam direction.

19. The system of claim 11, wherein determining the design of the range modulating device comprises converting the medical image into printer instructions.

20. The system of claim 19, wherein the printer instructions include fused deposition modeling (FDM) instructions.

21. The system of claim 11, wherein determining the design of the range modulating device comprises modeling density as a ratio of filament to voxel volume to emulate attenuation profiles for each voxel.

22. A method comprising:
assembling a composite range modulator by stacking a first range modulating device and a second range modulating device; and
performing radiotherapy on a patient by inserting the composite range modulator into a beam path between a radiation source and the patient.

23. The method of claim 22, wherein assembling the composite range modulator comprises stacking parallel to a particle beam direction, perpendicular to the particle beam direction, or parallel and perpendicular to the particle beam direction.

24. A range modulating device produced by the method of claim 1, the range modulating device comprising a plurality of spikes of energy shifting material having non-uniform density.
